(19) United States
(12) Patent Application Publication
(10) Pub. No.: US 2023/0138501 A1
(43) Pub. Date: May 4, 2023

(54) UV-light emitting air filtration mask

(71) Applicant: AIR Industries LLC, Metairie, LA (US)

(72) Inventors: Justin Hartenstein, Metairie, LA (US); Troy Organo, Metairie, LA (US); Alvin B Ricouard, New Orleans, LA (US)

(73) Assignee: AIR Industries LLC, Metairie, LA (US)

(21) Appl. No.: 17/907,614

(22) PCT Filed: Mar. 25, 2021
(86) PCT No.: PCT/US2021/024027

§ 371 Date: Sep. 28, 2022

Related U.S. Application Data
(60) Provisional application No. 63/006,950, filed on Apr. 8, 2020.
(60) Provisional application No. 63/015,069, filed on Apr. 24, 2020.
(60) Provisional application No. 63/032,838, filed on Jun. 1, 2020.
(60) Provisional application No. 63/139,402, filed on Jan. 20, 2021.

(51) Int. Cl. A61L 9/20; A61L 9/01; A62B 23/02; A41D 13/11

(52) U.S. Cl. CPC A61L 9/20; A61L 9/01; A62B 23/02; A41D 13/1192; A61L 2209/12; A61L 2209/14

(57) ABSTRACT

Described herein is a disinfecting system for a face mask. The disinfecting system may comprise a housing, a power source, a switch, and at least one strap. The housing may comprise a facial mounting surface, at least one passage, and at least one ultraviolet irradiation component capable of emitting ultraviolet (UV) irradiation. The power source may be electrically connected to the at least one ultraviolet irradiation component with the switch electrically connected to the power source and also electrically connected to the at least one ultraviolet irradiation component. It is further described herein a kit for disinfecting a face mask comprising the disinfecting system and a covering.

### CROSS REFERENCES AND PRIORITIES

This application claims priority from U.S. Provisional Application No. 63/006,950 filed on 8 Apr. 2020, U.S. Provisional Application No. 63/015,069 filed on 24 Apr. 2020, U.S. Provisional Application No. 63/032,838 filed on 1 Jun. 2020, and U.S. Provisional Application No. 63/139,402 filed on 20 Jan. 2021, the teachings of each of which are incorporated by reference herein in their entirety.

### BACKGROUND

Existing personal protection masks are designed to remove particulates from incoming air. Commonly known as surgical masks, face masks, N95 respirators, or N100 respirators, these filter devices are made from cloth, fiber material, pleated fabric, or other filter media to remove particle material from the air that the user is inhaling. Exhaled air flows through the same filter method. However, such personal protection masks are not designed to disinfect or sanitize the particles, nor are the filters designed specifically to inactivate airborne viruses, pathogens, or microorganisms.

The need exists, therefore, for a personal protection mask which disinfects and/or sanitizes air being transferred through the material of the mask.

### SUMMARY

It is disclosed a disinfecting system for a face mask. The disinfecting system for a face mask may comprise a housing, a power source, a switch, and at least one strap.

The housing may comprise a facial mounting surface, at least one passage, and at least one ultraviolet irradiation component capable of emitting ultraviolet (UV) irradiation.

The power source may be electrically connected to the at least one ultraviolet irradiation component. The switch may be electrically connected to the power source and may be electrically connected to the at least one ultraviolet irradiation component.

The at least one strap may be configured to seal the facial mounting surface to a user's face. The at least one strap may be connected at a first end to a first portion of the housing, and may be connected at a second end to a second portion of the housing.

In certain embodiments, the at least one ultraviolet irradiation component may comprise at least one light emitting diode. The at least one light emitting diode may be selected from the group consisting of at least one UVC light emitting diode, at least one UVA light emitting diode, and combinations thereof. In some embodiments, the at least one ultraviolet irradiation component may also comprise a circuit board. In certain embodiments, the at least one ultraviolet irradiation component may further comprise a secondary cover.

In some embodiments, the power source may be a battery. In certain embodiments, the power source may be connected to the at least one strap. In some embodiments, the switch may be integrally connected to the power source.

Certain embodiments may further comprise a primary cover. When used, the primary cover may be connected to the housing. The primary cover, when used, may be adapted to fit over at least a portion of the housing which comprises the at least one passage and the at least one ultraviolet irradiation component. In some such embodiments, the primary cover and the housing may be a single integral component.

In some embodiments, the housing may further comprise an anti-microbial coating. Alternatively, or in addition to, the housing in certain embodiments may be manufactured of a material having anti-microbial properties.

In certain embodiments, the at least one ultraviolet irradiation component may be oriented within the housing such that, when the housing is attached to the user's face, the at least one ultraviolet irradiation component emits irradiation in a direction opposite the user's face.

Certain embodiments may further comprise a covering. When used, the covering may be adapted to fit over at least a portion of the housing which comprises the at least one passage and the at least one ultraviolet irradiation component. In certain embodiments, the covering may be integrally connected to the housing by an adhesive and/or by a fastener. Alternatively, or in addition to, the covering may comprise at least one secondary strap. In some embodiments, the housing may be integrally connected to the covering. In such embodiments, there may be no straps connected directly to the housing. The covering, when used, may be made of a material selected from the group consisting of cloth materials, fibrous materials, and foamed materials.

In certain embodiments, the system may further comprise a filter material configured to fit over or within the passage. In some such embodiments, the filter material may be contained within a removable cartridge. When used, the removable cartridge may be adapted to attach to a surface of the housing. The filter material, when used, may be constructed of a material selected from the group consisting of fibrous filter materials, foamed filter materials, cloth filter materials, and combinations thereof. When a filter material is used, the ultraviolet irradiation component may be oriented within the housing such that at least a portion of the ultraviolet (UV) irradiation emitted from the ultraviolet irradiation component is directed towards the filter material.

It is also disclosed a kit for disinfecting a face mask. The kit may comprise a sub-mask assembly, and a covering. The sub-mask assembly may comprise a housing, a switch, and at least one strap.

The housing may comprise a facial mounting surface, at least one passage, and at least one ultraviolet irradiation component capable of emitting ultraviolet (UV) irradiation. The switch may be electrically connected to the at least one ultraviolet irradiation component. The covering may be adapted to fit over at least a portion of the housing which comprises the at least one passage and the at least one ultraviolet irradiation component.

The at least one strap may be configured to seal the facial mounting surface to a user's face. The at least one strap may be connected at a first end to a first portion of the housing and at a second end to a second portion of the housing.

In some embodiments of the kit, the at least one ultraviolet irradiation component may comprise at least one light emitting diode. The at least one light emitting diode may be selected from the group consisting of at least one UVC light emitting diode, at least one UVA light emitting diode, and combinations thereof. In some embodiments, the at least one ultraviolet irradiation component may also comprise a circuit board. In certain embodiments, the at least one ultraviolet irradiation component may further comprise a secondary cover.

Certain embodiments of the kit may further comprise a primary cover. When used, the primary cover may be connected to the housing. The primary cover, when used, may be adapted to fit over at least a portion of the housing which comprises the at least one passage and the at least one ultraviolet irradiation component. In some such embodiments, the primary cover and the housing may be a single integral component.

In certain embodiments of the kit, the housing may further comprise an anti-microbial coating. Alternatively, or in addition to, the housing in certain embodiments may be manufactured of a material having anti-microbial properties.

In some embodiments of the kit, the at least one ultraviolet irradiation component may be oriented within the housing such that, when the housing is attached to the user's face, the at least one ultraviolet irradiation component emits irradiation in a direction opposite the user's face.

In certain embodiments of the kit, the covering may comprise at least one secondary strap. The covering may be made of a material selected from the group consisting of cloth materials, fibrous materials, and foamed materials.

Certain embodiments of the kit may further comprise a power source which may be electrically connected to the at least one ultraviolet irradiation component. In some such embodiments, the switch may be electrically connected to the power source. The power source may be a battery. The power source may further be connected to the at least one strap. In some embodiments, the switch may be integrally connected to the power source.

Some embodiments of the kit may further comprise a filter material. The filter material may be configured to fit over or within the passage. In some such embodiments, the filter material may be contained within a removable cartridge. When used, the removable cartridge may be adapted to attach to a surface of the housing. The filter material, when used, may be constructed of a material selected from the group consisting of fibrous filter materials, foamed filter materials, cloth filter materials, and combinations thereof. When a filter material is used, the ultraviolet irradiation component may be oriented within the housing such that at least a portion of the ultraviolet (UV) irradiation emitted from the ultraviolet irradiation component is directed towards the filter material.

### BRIEF DESCRIPTION OF FIGURES

FIG. 1 depicts a partially exploded perspective view of one embodiment of a disinfecting system for a face mask.

FIG. 2 depicts an assembled version of the embodiment of a disinfecting system for a face mask shown in FIG. 1.

FIG. 3 depicts the disinfecting system for a face mask of FIG. 1 attached to a user's face.

FIG. 4 depicts the disinfecting system for a face mask of FIG. 3 with a covering placed over the disinfecting system.

FIG. 5 depicts the disinfecting system and covering of FIG. 4 with an illuminated ultraviolet irradiation component.

FIG. 6 depicts a partially exploded perspective view of one embodiment of a disinfecting system for a face mask.

### DETAILED DESCRIPTION

Disclosed herein is a disinfecting system for a face mask and a kit for disinfecting a face mask. The disinfecting system and kit are described below with reference to the Figures. As described herein and in the claims, the following numbers refer to the following structures as noted in the Figures.
10 depicts a user's face.
50 depicts a sub-mask assembly.
100 depicts a housing.
102 depicts a first portion of the housing.
104 depicts a second portion of the housing.
110 depicts a facial mounting surface.
120 depicts a passage.
130 depicts an ultraviolet irradiation component.
132 depicts a light emitting diode.
134 depicts a circuit board.
136 depicts a secondary cover.
140 depicts a mounting surface.
150 depicts a hole (in the housing).
200 depicts a covering.
210 depicts a secondary strap.
300 depicts a power source.
310 depicts a wire.
400 depicts a switch
500 depicts a strap.
510 depicts a first end of the strap.
520 depicts a second end of the strap.
600 depicts a primary cover.
610 depicts a hole (in the primary cover).
700 depicts a filter material.
710 depicts a removable cartridge.

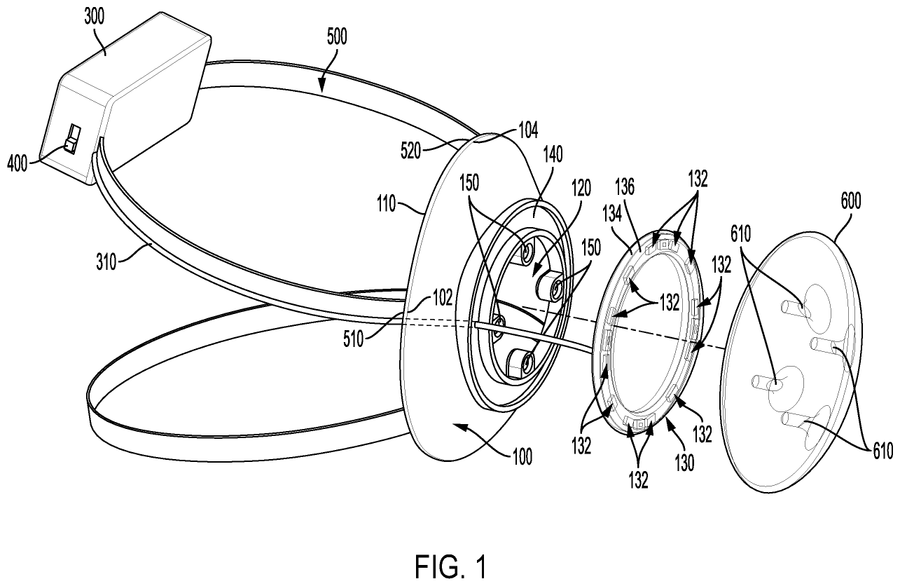

FIG. 1 depicts a partially exploded perspective view of one embodiment of a disinfecting system for a face mask. As shown in FIG. 1, the disinfecting system for a face mask may comprise a housing (100), a power source (300), a switch (400), and at least one strap (500).

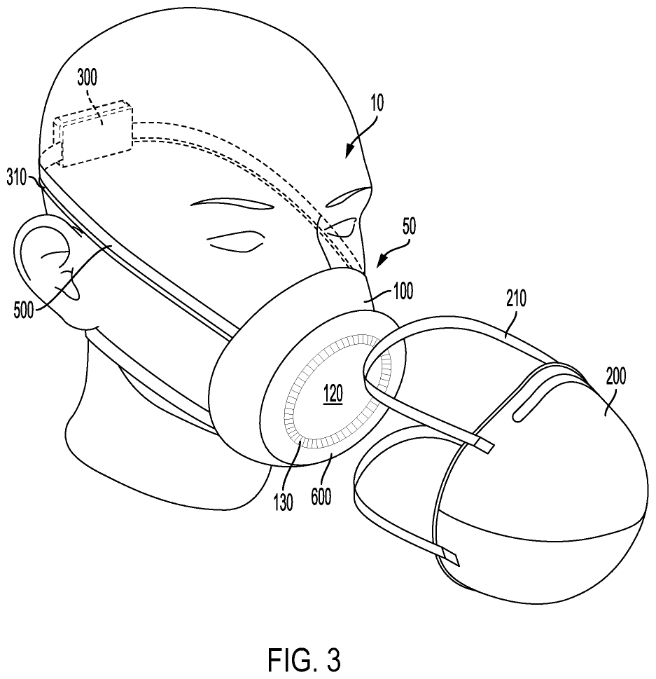

As shown in FIG. 1, the housing (100) may comprise a facial mounting surface (110). The facial mounting surface being a surface of the housing which—when in use—is in direct contact with the user's face (10 as shown in FIG. 3). The housing may also comprise at least one ultraviolet irradiation component (130). Preferably, the at least one ultraviolet irradiation component is capable of emitting irradiation in the form of light within the ultraviolet (UV) spectrum (also known as ultraviolet (UV) light) as described herein. As the at least one ultraviolet irradiation component emits light—albeit in the ultraviolet (UV) spectrum—the ultraviolet irradiation component may sometimes be referred to as a lighting component with the terms “ultraviolet irradiation component” and “lighting component” intended to each refer to the same structure. The at least one ultraviolet irradiation component may be attached to the housing on a mounting surface (140). The mounting surface is preferably located opposite the facial mounting surface.

The housing (100) may be fabricated from any number of flexible materials. The preferred material being a rubber or polymer composition. Preferred polymer compositions include flexible silicones, flexible epoxies, and flexible polyaspartics. The preferred manufacturing method for fabricating the housing is injection molding. In certain embodiments, the housing may further comprise an anti-microbial coating. Alternatively, or in addition to an anti-microbial coating, the housing may be fabricated of a material having anti-microbial properties and/or may include an anti-microbial additive. Non-limiting examples of compounds which may be used as an anti-microbial coating and/or anti-microbial additive include silver infused nanoparticles, copper infused nanoparticles, zinc containing antimicrobials, and N-butyl-1, 2-benzisothiazolin-3-one. A preferred zinc containing antimicrobial is zinc 2-pyridinethiol-1-oxide available from Lonza Group Ltd. of Basel Switzerland and is commonly sold under the trademark Zinc Omadine™, N-butyl-1, 2-benzisothiazolin-3-one is also available from Lonza Group Ltd. of Basel Switzerland and is commonly sold under the trademark Vanquish™ 100.

In some embodiments, the housing (100) may be fabricated from a rigid material such as rigid polymers or metals. In such embodiments, there may be an additional material attached to the housing along the perimeter of the facial mounting surface (110) which is made of a flexible material such as flexible rubber or polymer compositions. This additional material may assist in sealing the housing to the user's face and also improve user comfort. Such additional material may also be used in conjunction with a housing made of a flexible material as described herein.

FIG. 1 also shows the housing (100) comprising at least one passage (120). The at least one passage allows inhaled and exhaled air to pass through the housing. In certain embodiments there may be a single passage, while in other embodiments there may be a plurality of passages. The number, size, and shape of the passages is not considered important. However, it is preferred that the number, size, and shape of passages are each selected to allow sufficient airflow to and from the user to allow the user to maintain respiratory functions. This may be achieved by having a single large passage as shown in FIG. 1, or by having a number of smaller passages.

FIG. 1 further shows the ultraviolet irradiation component (130). As shown in FIG. 1, the ultraviolet irradiation component may comprise at least one light emitting diode (132)—also referred to herein as an LED. The light emitting diode(s) may be designed to create a wavelength of light having disinfecting characteristics. This type of disinfecting light is commonly known as ultraviolet germicidal irradiation (UVGI). UVGI is a disinfection method that uses short-wavelength ultraviolet A (UVA), ultraviolet B (UVB), and/or ultraviolet C (UVC) light to kill or inactivate microorganisms by destroying nucleic acids which, in turn, disrupts their DNA, rendering them inactive by leaving these cells unable to perform vital cellular functions.

The UVGI light produced by the light emitting diode(s) may aid in sterilization of air being inhaled to and exhaled out of the face mask as well as neutralizing contaminants trapped in the face mask itself. The irradiation of the UVGI light utilizes photons to disinfect the inhaled air. UVGI light damages a pathogen's DNA or RNA, which prevents it from replicating and infecting the body if inhaled. In addition to disinfecting inhaled or exhaled air, the UVGI light emitting diode(s) may also sterilize the filter material of the face mask itself.

Both the inhaled air and exhaled air may be exposed to the ultraviolet irradiation from the UVGI light emitting diode(s). UV light is electromagnetic radiation with wavelengths shorter than visible light, but longer than X-rays. UV can be separated into various ranges, with short-wavelength UVC generally considered to be “germicidal UV”. Wavelengths between about 200 nm and 300 nm are strongly absorbed by nucleic acids. The absorbed energy can result in defects including pyrimidine dimers. These dimers can prevent replication or prevent the expression of necessary proteins, resulting in the death or inactivation of the organism. However, light emitting diodes which emit UV light in a range selected from the group consisting of between 100 to 400 nm wavelengths, between 100 and 300 nm wavelengths, between 100 and 200 nm wavelengths, between 200 and 400 nm wavelengths, between 200 and 300 nm wavelengths, and between 300 and 400 nm wavelengths may also be utilized.

The at least one light emitting diode may be selected from the group consisting of at least one UVC light emitting diode, at least one UVA light emitting diode, and combinations thereof. In certain embodiments, the ultraviolet irradiation component may further comprise a circuit board (134), wherein at least one of the light emitting diode(s) may be electrically connected to the circuit board. Preferably, each of the light emitting diode(s) is electrically connected to the circuit board.

The light emitting diodes may be arranged in a pattern designed as shown in the Figures to expose both the air and filter inside the face mask to the UVGI light. The design may comprise a plurality of light emitting diode(s) and may have resistors and other electrical components integrated as well—any number of which may be electrically connected to the circuit board (134). The circuit board may preferably be connected to the power source (300) via the wire (310).

In some embodiments, there may be a secondary cover (136) over the ultraviolet irradiation component (130) as shown in FIG. 1. The secondary cover may encapsulate the various components (LED(s), circuit board, resistors and other electrical components) of the ultraviolet irradiation component. Preferably, the secondary cover is made of a glass or polymer material. Examples of glass materials may include quartz glass and sapphire glass. Examples of polymer materials may include epoxies, silicones, thermosets, and thermoplastics with the preferred polymer material being a moldable optical silicone elastomer or an aliphatic urethane. Preferably, the polymer material is UV-light stable. The epoxies—when used—may be selected from two-part epoxies and one-part epoxies, and may be either heat curable or UV curable. Preferably, the polymer material comprises a non-conductive resin. In some applications, the encapsulation of the ultraviolet irradiation component may be referred to as conformal coating in which a thin layer of the polymer material conforms to the contours of the circuit board and/or light emitting diodes to protect the various electrical components.

The material of the secondary cover (136) is preferably transparent to visible light and/or ultraviolet light, although embodiments may exist where the secondary cover is partially or fully opaque. As used herein, “transparent” may be defined as a material having a total transmittance (incident light−transmitted light) of at least 90.0% with at least 95.0% being preferred, at least 97.5% being more preferred, at least 99.0% being still more preferred, and 100% being most preferred. As used herein, “opaque” may be defined as a material having a total transmittance (incident light−transmitted light) of no more than 10.0% with no more than 7.5% being preferred, no more than 5.0% being more preferred, and no more than 2.5% being most preferred. The material may also include features such as coloring, opaque materials, texturing, or diffusing materials.

Also shown in FIG. 1 is at least one strap (500). As shown in FIG. 1, the strap may have a first end (510) connected to a first portion of the housing (102) and a second end (520) connected to a second portion of the housing (104). While FIG. 1 shows two separate straps, each of which is designed to wrap around the user's head as shown in FIG. 3, other embodiments may exist. For example, embodiments may exist in which there is a single strap designed to wrap around the user's head. Other embodiments may comprise a pair of straps connected to opposing sides of the housing with each strap designed to wrap around an opposing ear of the user.

The at least one strap may be fabricated of a material such as rubber or elastic cord having a reversible deformation which allows the material to stretch and partially or fully return to its original state. Such material may assist in partially or fully sealing the facial mounting surface (110) to the user's face (10) by pulling the housing towards the user's face.

FIG. 1 also shows a power source (300). The power source may be electrically connected to the at least one ultraviolet irradiation component (130). Such connection may occur via a wire (310) which is connected at one end to the power source and at a second end to the at least one ultraviolet irradiation component. Preferably, the power source is a battery. While the power source shown in the Figures is attached to one of the straps (500), one of ordinary skill will recognize that the power source may be attached to any one of the components including a surface of the housing (100), and a surface of the (optional) primary cover (600). In certain embodiments, the power source may further comprise a recharging mechanism such as a solar cell electrically connected to the battery. Another example of a recharging mechanism may include an electrical connection which can be plugged into a standard wall outlet via a cable.

Finally, FIG. 1 shows a switch (400). The switch may be electrically connected to the power source (300) and also may be electrically connected to the at least one ultraviolet irradiation component (130). The switch may be configured to allow a user to turn the at least one ultraviolet irradiation component on and off by changing the switch from an on position in which the switch closes the electrical circuit between the power source and the at least one ultraviolet irradiation component allowing electricity from the power source to flow to the at least one ultraviolet irradiation component, to an off position in which the switch opens the electrical circuit between the power source and the at least one ultraviolet irradiation component preventing electricity from the power source from flowing to the at least one ultraviolet irradiation component.

In some embodiments, the switch (400) may be integrally connected to the power source (300) within a power source housing as shown in the Figures. In other embodiments, the switch may be a stand-alone switch attached to one or more components selected from the group consisting of the housing (100), the (optional) primary cover (600), or one or more of the straps (500). In such embodiments, the wire (310) may comprise two separate wires—one of which electrically connects the power source to the switch while the other electrically connects the switch to the ultraviolet irradiation component (130).

FIG. 1 also shows an optional primary cover (600). The primary cover is considered optional as it may not be present in all embodiments. When used, the primary cover disburses at least a portion of the irradiation emitted from the light emitting diode(s) (132), and may also increase the residence time of inhaled and/or exhaled air passing to and from the user's nose and mouth. This increased residence time may increase the ability for the ultraviolet irradiation emitted from the light emitting diodes to inactivate bacterial and/or viral cells in the inhaled or exhaled air.

The primary cover (600) may be fabricated from any number of materials. For example, the primary cover may be made of a glass or polymer material. Examples of glass materials may include quartz glass and sapphire glass. Examples of polymer materials may include epoxies, silicones, thermosets, and thermoplastics. The preferred material being a partially or fully transparent rigid plastic material. Preferred examples include acrylic materials with poly(methyl methacrylate (PMMA) being a most preferred example. In certain embodiments, the primary cover may be fabricated of a material which includes a UV blocker. One example of a UV blocker is a liquid hindered amine light stabilizer with the preferred liquid hindered amine light stabilizer being Tinuvin® 292 available from BASF Corporation, Charlotte, N.C., U.S.A. Another example of a UV blocker is a polyaspartic coating. In certain embodiments, a polyaspartic material having UV blocking properties may be used to fabricate all or a portion of the primary cover instead of or in addition to PMMA. Preferred manufacturing methods include injection molding. The primary cover may be connected to the housing by passing one or more fasteners—such as a screw, bolt, or clip, through a hole (610) in the primary cover and into a corresponding hole (150) in the housing. Alternatively, the primary cover and the housing (100) may be manufactured as a single integral component such as by injection molding.

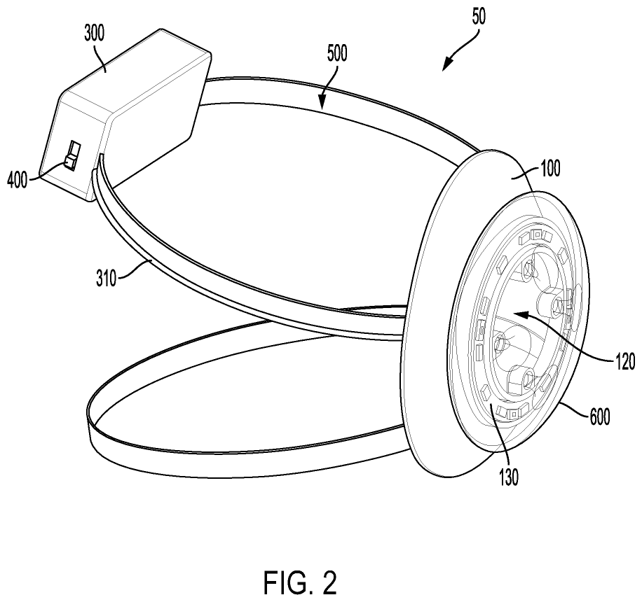

FIG. 2 depicts an assembled perspective view of the embodiment of a disinfecting system for a face mask shown in FIG. 1. As shown in FIG. 2, once assembled the ultraviolet irradiation component (130) is connected to the housing within the mounting surface (140 as shown in FIG. 1). This connection may occur via an adhesive, or via a fastener such as one or more clips, one or more screw, or combinations of different fasteners and/or adhesives. Covering the ultraviolet irradiation component as shown in FIG. 2 may be the primary cover (600).

In FIG. 3, an embodiment of a disinfecting system for a face mask has been placed over a user's face (10). As shown in FIG. 3, the ultraviolet irradiation component (130) may be oriented within the housing (100) such that, when the housing is attached to the user's face, the ultraviolet irradiation component emits irradiation in a direction opposite the user's face and towards the covering (200). This is done in order to minimize or prevent the exposure of the user's skin to the UV irradiation emitted from the LED(s). Preferably, less than 10% of the irradiation emitted from the ultraviolet irradiation component is directed toward the user's face with less than 5% being more preferred, less than 1% being even more preferred, less than 0.1% being still more preferred, and 0.0% being most preferred.

In some embodiments, all or a portion of the irradiation emitted from the ultraviolet irradiation component (130) may be directed towards a reflective surface. The reflective surface may be a surface of the housing (100), a surface of the primary cover (600), or an additional surface attached to the housing. A preferred reflective surface is a surface which has been treated with evaporated aluminum. By directing all or a portion of the irradiation emitted from the ultraviolet irradiation component towards a reflective surface, the light may reflect off of the surface thereby increasing the dispersion of the light. This can increase the surface area of the housing, primary cover, and covering (200) which is exposed to the emitted irradiation, and also increase the duration of time which particles in the air are exposed to the emitted irradiation—both of which are thought to improve the ability of the light to neutralize bacterial and/or viral particles.

In certain embodiments, the housing (100) may include a circuitous pathway. A circuitous pathway as used herein and in the claims describes a tube, passage, conduit, or the like which increases the length of the pathway that inbound air is directed through when passing through the filter sub-mas assembly (50). The circuitous pathway may take many forms with a serpentine pathway and a spiral pathway being considered non-limiting examples of preferred pathways. Preferably all or a majority of the inner surface of the circuitous pathway will be exposed to the irradiation emitted from the ultraviolet irradiation component (130) with at least 51% of the inner surface of the circuitous pathway being exposed to the irradiation emitted from the first ultraviolet irradiation component being preferred, at least 75% being more preferred, at least 90% being still more preferred, and at least 99% being most preferred. The circuitous pathway is preferably an integral component of the housing such as by injection molding the circuitous path as part of the housing. However, embodiments may exist in which the circuitous pathway is a separate component which is connected to an inlet of the housing.

FIG. 3 also introduces a covering (200)—which is also referred to herein as a face mask. The covering may be a standard face mask available from any variety of suppliers. The covering may be made of any number of materials commonly used for face masks. Such materials may be selected from the group consisting of cloth materials, fibrous materials, and foamed materials.

As shown in FIG. 3, the covering (200) may comprise at least one secondary strap (210). The secondary strap(s) shown in FIG. 3 are designed as a pair of straps connected to opposing sides of the covering with each secondary strap designed to wrap around an opposing ear of the user. Alternatively, the secondary strap(s) may be designed to wrap around the user's head in much the same manner as the straps (500) attached to the housing in the embodiment shown in FIG. 1 and FIG. 2.

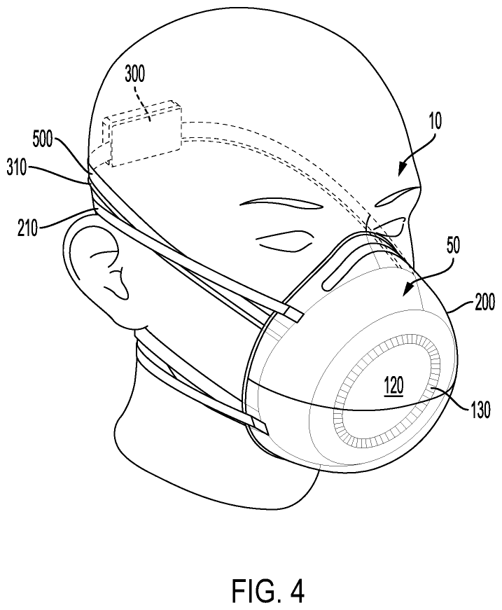

FIG. 4 shows the covering (200) placed onto the user's face (10). Preferably, when placed onto the user's face, the covering is adapted to fit over at least a portion of the housing which comprises the at least one passage (120) and the at least one ultraviolet irradiation component (130).

While FIG. 4 shows the covering (200) as a separate piece which can quickly be attached over or removed from the sub-mask assembly (50) by removing the secondary strap (210) from the user's face, other embodiments may exist. For instance, in some embodiments, the covering may be integrally connected to the sub-mask assembly (50) such as by an adhesive or by way of a fastener such as a clip, screw, rivet, or staple.

Alternatively, the housing (100) may be integrally connected to the covering (200) such that there are no straps (500) connected directly to the housing. In such embodiments, the secondary strap(s) (210) of the covering may be the only straps used for connected the covering to the user's face. In some such embodiments, there may be a secondary covering connected to the housing on a surface opposite that of the ultraviolet irradiation component, said secondary covering providing for improved sealing to the user's face as well as additional particulate filtration.

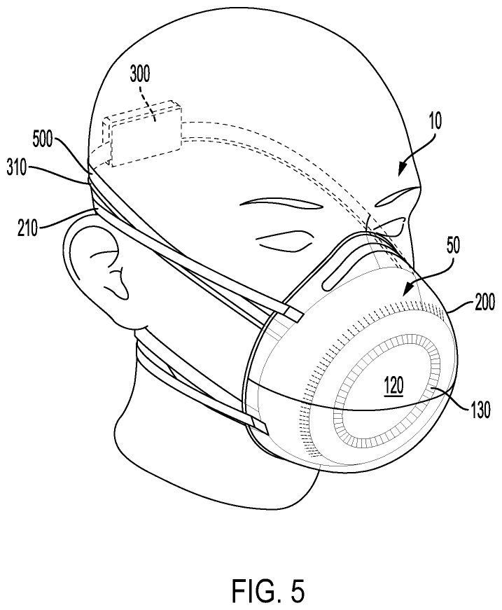

FIG. 5 depicts the disinfecting system and the covering (200) with the ultraviolet irradiation component (130) illuminated. Illuminating the ultraviolet irradiation component may involve turning the switch (400) to an “on” position which closes the circuit between the power source (300) and the ultraviolet irradiation component (130). As described herein, illuminating the ultraviolet irradiation component generates UVGI light which may deactivate bacteria and/or viruses being inhaled and/or exhaled by the user, and/or which have become trapped in the covering material.

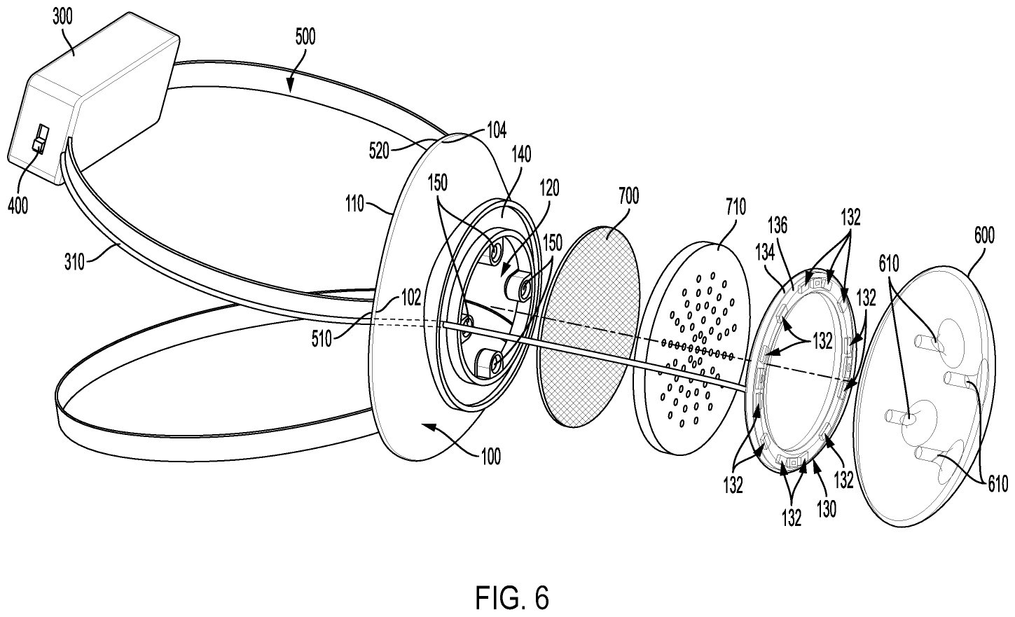

FIG. 6 depicts an alternative embodiment comprising a filter material (700). The filter material—when used—may be configured to fit over or within the passage (120) of the housing (100) to filter particles, bacteria, and/or viruses from the air passing through the passage. The filter material may be constructed of a material selected from the group consisting of fibrous filter materials, foamed filter materials, cloth filter materials, and combinations thereof.

In some embodiments, the filter material (700) may be contained within a removable cartridge (710). The removable cartridge (710) may be designed to attach to a surface of the housing (100) to allow the filter material to be easily removed and replaced. While the filter material and removable cartridge shown in FIG. 6 are attached to an outer surface of the housing, one of ordinary skill will easily recognize that the filter material and removable cartridge may also be attached to an inner surface of the housing.

When the filter material (700) is used, it is preferred that the ultraviolet irradiation component (130) be oriented within the housing such that all or at least a portion of the irradiation emitted from the ultraviolet irradiation component is directed towards the filter material.

The filter material (700) may eliminate the need for a covering (200). However, in certain embodiments, the filter material may be used in conjunction with a covering.

It is also envisioned a kit for disinfecting a face mask. The kit may describe a sub-mask assembly (50 as shown in FIGS. 3 to 5) with the sub-mask assembly comprising the housing (100) with all of its components described herein, switch (400), and at least one strap (500) as described herein. In certain embodiments, the kit may also comprise a power source (300) as described herein, although the kit may be provided without a power source allowing the end user to purchase and install a separate power source. In such embodiments, the sub-mask assembly may include a secondary housing capable of receiving a power source and electrically connected to the lighting element (130) and switch.

In some embodiments, the sub-mask assembly may also comprise a primary cover (600) as described herein. In certain embodiments, the sub-mask assembly may also comprise a filter material (700) as described herein with or without a removable cartridge (710). The kit may also comprise a covering (200) of the type described herein with the covering adapted to fit over at least a portion of the housing which comprises the at least one passage and the at least one ultraviolet irradiation component.

1. A disinfecting system for a face mask comprising:
a housing (100) comprising:
a facial mounting surface (110),
at least one passage (120), and
at least one ultraviolet irradiation component (130) capable of emitting ultraviolet (UV) irradiation;
a power source (300) electrically connected to the at least one ultraviolet irradiation component;
a switch (400) electrically connected to the power source and electrically connected to the at least one ultraviolet irradiation component; and
at least one strap (500) configured to seal the facial mounting surface to a user's face (10), said strap connected at a first end (510) to a first portion of the housing (102) and at a second end (520) to a second portion of the housing (104).

2. The disinfecting system for a face mask of claim 1, wherein the at least one ultraviolet irradiation component comprises at least one light emitting diode (132) selected from the group consisting of at least one UVC light emitting diode, at least one UVA light emitting diode, and combinations thereof.

3. The disinfecting system for a face mask of claim 1, wherein the at least one ultraviolet irradiation component comprises a circuit board (134).

4. The disinfecting system for a face mask of claim 1, wherein the at least one ultraviolet irradiation component comprises a secondary cover (136).

5. The disinfecting system for a face mask of claim 1, wherein the power source is a battery.

6. (canceled)

7. The disinfecting system for a face mask of claim 1, wherein the switch is integrally connected to the power source.

8. The disinfecting system for a face mask of claim 1, further comprising a primary cover (600) connected to the housing, said primary cover adapted to fit over at least a portion of the housing which comprises the at least one passage and the at least one ultraviolet irradiation component.

9. The disinfecting system for a face mask of claim 8, wherein the primary cover and the housing are a single integral component.

10. The disinfecting system for a face mask of claim 1, wherein the housing further comprises an anti-microbial coating.

11. The disinfecting system for a face mask of claim 1, wherein the housing is manufactured of a material having anti-microbial properties.

12. The disinfecting system for a face mask of claim 1, wherein the at least one ultraviolet irradiation component is oriented within the housing such that, when the housing is attached to the user's face, the at least one ultraviolet irradiation component emits ultraviolet irradiation in a direction opposite the user's face.

13. The disinfecting system for a face mask of claim 1, further comprising a covering (200) adapted to fit over at least a portion of the housing which comprises the at least one passage and the at least one ultraviolet irradiation component.

14. The disinfecting system for a face mask of claim 13, wherein the covering is integrally connected to the housing by an adhesive and/or by a fastener.

15. The disinfecting system for a face mask of claim 13, wherein the covering comprises at least one secondary strap (210).

16. (canceled)

17. The disinfecting system for a face mask of claim 13, wherein the covering is made of a material selected from the group consisting of cloth materials, fibrous materials, and foamed materials.

18. The disinfecting system for a face mask of claim 1, further comprising a filter material (700) configured to fit over or within the passage.

19. The disinfecting system for a face mask of claim 18, wherein the filter material is contained within a removable cartridge (710), and the removable cartridge is adapted to attached to a surface of the housing.

20. The disinfecting system for a face mask of claim 18, wherein the filter material is constructed of a material selected from the group consisting of fibrous filter materials, foamed filter materials, cloth filter materials, and combinations thereof.

21. The disinfecting system for a face mask of claim 18, wherein the ultraviolet irradiation component is oriented within the housing such that at least a portion of the ultraviolet (UV) irradiation emitted from the ultraviolet irradiation component is directed towards the filter material.

22. A kit for disinfecting a face mask comprising:
a sub-mask assembly (50) comprising:
a housing (100) comprising:
a facial mounting surface (110),
at least one passage (120), and
at least one ultraviolet irradiation component (130) capable of emitting ultraviolet (UV) irradiation,
a switch (400) electrically connected to the at least one ultraviolet irradiation component, and
at least one strap (500) configured to seal the facial mounting surface to a user's face (10), said strap connected at a first end (510) to a first portion of the housing (102) and at a second end (520) to a second portion of the housing (104); and
a covering (200) adapted to fit over at least a portion of the housing which comprises the at least one passage and the at least one ultraviolet irradiation component.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)
